(19) United States
(12) Patent Application Publication
(10) Pub. No.: US 2022/0241180 A1
(43) Pub. Date: Aug. 4, 2022

(54) COMPOSITION COMPRISING SORBITOL OR XYLITOL, AND A GELLING AGENT

(71) Applicant: COSMETIC WARRIORS LIMITED, Poole, Dorset (GB)

(72) Inventors: Mark CONSTANTINE, Poole, Dorset (GB); Margaret Joan CONSTANTINE, Poole, Dorset (GB); Helen Elizabeth AMBROSEN, Poole, Dorset (GB); Rowena Jacqueline BIRD, Poole, Dorset (GB); Alessandro COMMISSO, Poole, Dorset (GB); Gary SHEARS, Poole, Dorset (GB)

(21) Appl. No.: 17/612,512

(22) PCT Filed: May 22, 2020
(86) PCT No.: PCT/GB2020/051254

§ 371 Date: Nov. 18, 2021

(30) Foreign Application Priority Data
May 22, 2019 (GB) 1907225.5

(51) Int. Cl. A61K 8/9717; A61K 8/34; A61K 8/25; A61K 8/9711; A61K 8/02; A61Q 11/00; A61K 8/44

(52) U.S. Cl. CPC A61K 8/9717; A61K 8/345; A61K 8/25; A61K 2800/48; A61K 8/0216; A61Q 11/00; A61K 8/442; A61K 8/9711

(57) ABSTRACT

A composition includes (i) a sugar alcohol selected from sorbitol, xylitol, and mixtures thereof; (ii) water in an amount of from about 20% to about 60% by weight of the composition; and (iii) a gelling agent.

### FIELD OF THE INVENTION

The present invention relates to a product (preferably for use as a cosmetic), a process for producing said product, and a product prepared by the process.

### BACKGROUND TO THE INVENTION

The present invention relates to products particularly those for use in contact with the human body, preferably for use as an oral care product, for example as a composition for cleaning teeth (i.e. a toothpaste replacement). The present invention relates to the increasing demand for fresh and natural cosmetics, which are free from preservatives and in packaging which has a low environmental impact or even packaging-free.

The rise of cosmetics to the pinnacle of the modern beauty industry is not a recent phenomenon. Humans have been using moisturisers since the Mesolithic era (10,000 years ago). The Sumerians combined wine, tree oils and plant, animal or mineral materials before applying to the body. The ancient Egyptian civilisation is known to have used olive oil, sesame oil, myrrh resin as well as bitter almonds, honey and cardamom to care for the skin. Ancient civilisations have also made great efforts to take care of their teeth, using sticks and twigs to clean and also substances such as charcoal and soot to scour and whiten the teeth. Tooth decay could lead to health issues which were fatal. Exploration of Egyptian mummies has shown deaths from abscesses and such. Today, modern dentistry has made a high standard of oral care more accessible. However, personal care is still needed to maintain healthy teeth.

Modern cosmetic products are formulated to have a very long shelf-life. The shelf-life of a typical product is thirty one months. However, before products reach a retail environment, they are shipped and then stored in warehouses, sometimes for years before sale. Products may be labelled with a ‘best used by date’ but without an indication of when they were made. Consequently many formulations are assembled with longevity more in mind than effect.

Consumers are increasingly concerned with the ingredients used in their cosmetics. There have been numerous reports in the media linking various materials in cosmetics with increased risk of various diseases including cancer. One materials category that has been associated with these stories on numerous occasions is preservatives.

The purpose of cosmetic preservatives is to prevent the growth of microorganisms (including yeasts & moulds), which would have a detrimental influence on the effect or the appearance of the product as well as being a risk to human health. However excessive use of preservatives can be employed to increase shelf-life and therefore the profitability of cosmetics. In response to media or consumer pressure, the industry may switch from one preservative system to another; however it would be more desirable to provide a range of effective preservative-free cosmetics. By eliminating preservative systems from cosmetics, it would be possible to eliminate worries of consumers, such as bioaccumulation through repetitive use. It would also be possible to reduce the environmental impact of the cosmetic products. The use of preservatives in the manufacture of products and use by the consumer leads to the contamination of the environment by these materials.

Furthermore, cosmetic or toiletry products such as toothpastes are conventionally in a liquid or semi-liquid state (e.g. in the form of a paste or a low-viscosity gel) housed in a plastic and/or metal, non-recyclable and non-biodegradable tube. Such packaging will stay in landfill systems for many generations without fully degrading. Such toothpastes are not able to sustain their physical shape when unsupported by external means, and thus the user must apply the product by squeezing the paste out of the packaging in which it is contained. Conventional toothpastes also typically require preservatives in order to maintain their shelf-life over extended periods of storage.

The present invention seeks to provide a product (preferably a cosmetic product) which is free from preservatives, and which obviates the need for external packaging as it can substantially sustain its physical shape when unsupported by external means whilst being sufficiently soft that the user can remove a portion of the product, for example using an applicator or fingers, without needing to break or snap the whole product.

### SUMMARY OF THE INVENTION

According to a first aspect of the invention, there is provided a composition comprising (i) a sugar alcohol selected from the group consisting of sorbitol, xylitol, and mixtures thereof; (ii) water in an amount of from about 20% to about 60% by weight of the composition; and (iii) a gelling agent.

According to a second aspect of the invention, there is provided a process for the production of a composition comprising (i) a sugar alcohol selected from the group consisting of sorbitol, xylitol, and mixtures thereof; (ii) water in an amount of from about 20% to about 60% by weight of the composition; and (iii) a gelling agent; the process comprising the following steps:
(a) preparing a preparatory composition comprising water and a sugar alcohol selected from the group consisting of sorbitol, xylitol, and mixtures thereof;
(b) contacting the preparatory composition with a gelling agent; and
(c) introducing the resultant composition to a mould.

According to a third aspect of the invention, there is provided a product obtained or obtainable by a process for the production of a composition comprising (i) a sugar alcohol selected from the group consisting of sorbitol, xylitol, and mixtures thereof; (ii) water in an amount of from about 20% to about 60% by weight of the composition; and (iii) a gelling agent; the process comprising the following steps:
(a) preparing a preparatory composition comprising water and a sugar alcohol selected from the group consisting of sorbitol, xylitol, and mixtures thereof;
(b) contacting the preparatory composition with a gelling agent; and
(c) introducing the resultant composition to a mould.

As described herein, the invention provides a composition. Preferably, the composition is a cosmetic composition. As used herein, the term “cosmetic composition” means a composition that may be used to improve or maintain a user's appearance. The term “cosmetic composition” is not intended to limit the composition to those regulated under Cosmetics Directives or the like, but rather encompasses any composition used to improve or maintain a user's appearance. Thus, as used herein, the term “cosmetic composition” may be a skin care agent (such as, for example, moisturising agent, sunblock, sunscreen, skin cleansing composition, exfoliator, or the like), a hair care agent (such as, for example, a shampoo and/or conditioner, a hair mask, a hair dye, or the like) and/or an oral care agent (such as, for example, a tooth cleansing composition (toothpaste-like), a breath freshening agent, a mouthwash, or the like).

Throughout the specification, any reference to a “composition” may also refer to a “cosmetic composition”.

In some embodiments, the composition may be a pharmaceutical composition. It is noted that the composition may have a dual purpose or effect. For example, the composition may be regarded as a cosmetic composition as it maintains the appearance of the user, but may also have a secondary effect such as avoiding or treating tooth decay. Nevertheless, since the composition still maintains the appearance of a user, such composition may still be regarded as a cosmetic composition.

The term “free from preservatives” means that the composition contains any of the preservatives listed in Table 1 in amounts of no greater than the amounts specified. Preferably, the composition is entirely free from all of the preservatives listed in Table 1. Preferably, the composition is entirely free from all synthetic preservatives (including but not limited to those listed in Table 1).

TABLE 1

| | Present in an amount no greater than | |
|---|---|---|
| Preservative | (based on the total composition) | |
| Benzoic acid | 0.062% | w/w |
| Sodium benzoate | 0.062% | w/w |
| Salts of benzoic acid excluding sodium benzoate | 3 × 10⁻³ | % w/v |
| Propionic acid and salts thereof | 3.2 × 10⁻³ | % w/v |
| Salicylic acid and salts thereof | 1% | w/v |
| Sorbic acid and salts thereof | 0.4% | w/v |
| Formaldehyde | 0.01% | w/v |
| Paraformaldehyde | 0.01% | w/v |
| Biphenyl-2-ol and salts thereof | 1 × 10⁻⁵ | % w/v |
| Pyrithione zinc | 1.6 × 10⁻⁴ | % w/v |
| Inorganic sulphites | 1.6 × 10⁻⁵ | % w/v |
| Hydrogen sulphites | 1.6 × 10⁻⁵ | % w/v |
| Chlorobutanol | 0.25% | w/v |
| 4-Hydroxybenzoic acid and salts thereof and | 0.012% | w/v |
| esters thereof | | |
| Butyl 4-hydroxybenzoate and salts thereof | 0.012% | w/w |
| Propyl 4-hydroxybenzoate and salts thereof | 0.012% | w/w |
| 3-Acetyl-6-methylpyran-2,4(3H)-dione and | 1 × 10⁻³ | % w/v |
| salts thereof | | |
| Formic acid | 6 × 10⁻³ | % w/v |
| Sodium formate | 6 × 10⁻³ | % w/v |
| 3,3′-Dibromo-4,4′-hexamethylene | 6 × 10⁻⁵ | % w/v |
| dioxydibenzamidine and salts thereof | | |
| Thiomersal | 3.2 × 10⁻³ | % w/w |
| Phenylmercuric salts | 3 × 10⁻⁵ | % w/w |
| Undec-10-enoic acid and salts thereof | 2.5 × 10⁻³ | % w/v |
| 5-pyrimidinamine, 1,3-bis(2- | 6.6 × 10⁻⁴ | % w/w |
| ethylhexyl)hexahydro-5-methyl | | |
| 5-bromo-5-nitro-1,3-dioxane | 8 × 10⁻³ | % w/w |
| Bronopol | 8 × 10⁻³ | % w/w |
| 2,4-dichlorobenzyl alcohol | 0.0625% | w/w |
| 1-(4-chlorophenyl)-3-(3,4-dichlorophenyl)urea | 5 × 10⁻⁵ | % w/w |
| Chlorocresol | 5 × 10⁻⁵ | % w/w |
| 5-chloro-2-(2,4-dichlorophenoxy)phenol | 4 × 10⁻⁶ | % w/w |
| Chloroxylenol | 7 × 10⁻⁵ | % w/w |
| N,N″-methylenebis[N′-{3-(hydroxymethyl)-2,5- | 0.1% | w/v |
| dioxoimidazolidin-4-yl}urea] | | |
| Poly(hexamethylenebiguanide) hydrochloride | 3.1 × 10⁻³ | % w/v |
| 2-Phenoxyethanol | 5 × 10⁻⁴ | % w/v |
| Methenamine | 4 × 10⁻³ | % w/w |
| Methenamine 3-chloroallylochloride | 5 × 10⁻³ | % w/v |
| 1-(4-Chlorophenoxy)-1-(imidazole-1-yl)-3,3- | 6.3 × 10⁻⁵ | % w/v |
| dimethylbutan-2-one | | |
| 1,3-Bis (hydroxymethyl)-5,5- | 6.3 × 10⁻⁵ | % w/v |
| dimethylimidazolidine-2,4-dione | | |
| Benzyl alcohol | 0.0625% | w/v |
| 1-Hydroxy-4-methyl-6-(2,4,4-trimethylpentyl) | 1.6 × 10⁻⁵ | % w/v |
| 2-pyrindon | | |
| Monoethanolamine salt of 1-Hydroxy-4- | 1.6 × 10⁻⁵ | % w/v |
| methyl-6-(2,4,4-trimethylpentyl) 2-pyrindon | | |
| 2,2′-methylenebis(6-bromo-4-chlorophenol) | 2 × 10⁻³ | % w/v |
| 4-Isopropyl-m-cresol | 0.1% | w/v |
| 5-Chloro-2-methyl-isothiazol-3(2H)-one | 1.6 × 10⁻³ | % w/v |
| 2-methyl-isothiazol-3(2H)-one | 1.6 × 10⁻³ | % w/v |
| 2-Benzyl-4-chlorophenol | 0.1% | w/v |
| 2-Chloroacetamide | 6.25 × 10⁻³ | % w/v |
| N,N″-bis(chlorophenyl)-3,12-diamino- | 5 × 10⁻⁴ | % w/v |
| 2,4,11,13-tetraazatetradecadiamidine | | |
| digluconate of N,N″-bis(chlorophenyl)-3,12- | 5 × 10⁻⁴ | % w/v |
| diamino-2,4,11,13-tetraazatetradecadiamidine | | |
| diacetate of N,N″-bis(chlorophenyl)-3,12- | 5 × 10⁻⁴ | % w/v |
| diamino-2,4,11,13-tetraazatetradecadiamidine | | |
| dihydrochloride of N,N″-bis(chlorophenyl)- | 5 × 10⁻⁴ | % w/v |
| 3,12-diamino-2,4,11,13-tetraazatetradecadiamidine | | |
| 1-Phenoxypropanol-2-ol | 0.1% | w/v |
| Alkyl(C₁₂₋₂₂)trimethyl ammonium bromide | 6.1 × 10⁻³ | % w/v |
| Alkyl(C₁₂₋₂₂)trimethyl ammonium chloride | 6.1 × 10⁻³ | % w/v |
| 4,4-Dimethyl-1,3-oxazolidine | 3 × 10⁻⁶ | % w/v |
| N-(hydroxymethyl)-N-(dihydroxymethyl-1,3- | 3 × 10⁻⁵ | % w/v |
| dioxo-2,5-imidazolidinyl-4)-N′(hydroxymethyl) | | |
| urea | | |
| Benzenecarboximidamide, 4,4′-(1,6- | 0.116% | w/v |
| hexanediylbis (oxy))bis- and salts thereof | | |
| Glutaraldehyde (pentane-1,5-dial) | 5 × 10⁻⁴ | % w/v |
| 5-Ethyl-3,7-dioxa-1-azabicyclo[3.3.0] octane | 1.5 × 10⁻⁴ | % w/v |
| 3-(p-Chlorophenoxy)-propane-1,2 diol | 1.25% | w/v |
| Sodium hydroxymethylamino acetate | 2 × 10⁻⁵ | % w/v |
| Silver chloride | 3.12 × 10⁻³ | % w/v |
| Benzalkonium chloride | 4 × 10⁻³ | % w/v |
| Benzalkonium bromide | 4 × 10⁻³ | % w/v |
| Benzalkonium saccharinate | 4 × 10⁻³ | % w/v |
| Phenylmethoxy methanol | 3 × 10⁻⁵ | % w/v |
| 3-Iodo-2-propynylbutylcarbamate | 6.25 × 10⁻³ | % w/v |

In the process of the present invention, the process preferably excludes a step of addition one or more prohibited preservatives. The prohibited preservatives are those listed in Table 2. Preferably, the process excludes a step of addition of one or more of any synthetic preservatives.

TABLE 2

| | |
|---|---|
| Benzoic acid | 5-Chloro-2-methyl-isothiazol-3(2H)-one |
| Sodium benzoate | 2-methyl-isothiazol-3(2H)-one |
| Salts of benzoic acid excluding sodium benzoate | 2-Benzyl-4-chlorophenol |
| Propionic acid and salts thereof | 2-Chloroacetamide |
| Salicylic acid and salts thereof | N,N″-bis(chlorophenyl)-3,12-diamino- |
| | 2,4,11,13-tetraazatetradecadiamidine |
| Sorbic acid and salts thereof | digluconate of N,N″-bis(chlorophenyl)-3,12- |
| | diamino-2,4,11,13-tetraazatetradecadiamidine |
| Formaldehyde | diacetate of N,N″-bis(chlorophenyl)-3,12- |
| | diamino-2,4,11,13-tetraazatetradecadiamidine |
| Paraformaldehyde | dihydrochloride of N,N″-bis(chlorophenyl)- |
| | 3,12-diamino-2,4,11,13-tetraazatetradecadiamidine |
| Biphenyl-2-ol and salts thereof | 1-Phenoxypropanol-2-ol |
| Pyrithione zinc | Alkyl(C₁₂₋₂₂)trimethyl ammonium bromide |
| Inorganic sulphites | Alkyl(C₁₂₋₂₂)trimethyl ammonium chloride |
| Hydrogen sulphites | 4,4-Dimethyl-1,3-oxazolidine |
| Chlorobutanol | N-(hydroxymethyl)-N-(dihydroxymethyl-1,3- |
| | dioxo-2,5-imidazolidinyl-4)-N′(hydroxymethyl) urea |
| 4-Hydroxybenzoic acid and salts thereof and | Benzenecarboximidamide, 4,4′-(1,6- |
| esters thereof | hexanediylbis (oxy))bis- |
| 3-Acetyl-6-methylpyran-2,4(3H)-dione and | Glutaraldehyde (pentane-1,5-dial) |
| salts thereof | |
| Formic acid | 5-Ethyl-3,7-dioxa-1-azabicyclo[3.3.0] octane |
| Sodium formate | 3-(p-Chlorophenoxy)-propane-1,2 diol |
| Methenamine 3-chloroallylochloride | Sodium hydroxymethylamino acetate |
| 1-(4-Chlorophenoxy)-1-(imidazole-1-yl)-3,3- | Silver chloride |
| dimethylbutan-2-one | |
| 1,3-Bis (hydroxymethyl)-5,5- | Benzalkonium chloride |
| dimethylimidazolidine-2,4-dione | |
| Benzyl alcohol | Benzalkonium bromide |
| 1-Hydroxy-4-methyl-6-(2,4,4-trimethylpentyl) | Benzalkonium saccharinate |
| 2-pyrindon | |
| Monoethanolamine salt of 1-Hydroxy-4- | Phenylmethoxy methanol |
| methyl-6-(2,4,4-trimethylpentyl) 2-pyrindon | |
| 2,2′-methylenebis(6-bromo-4-chlorophenol) | 3-Iodo-2-propynylbutylcarbamate |
| 4-Isopropyl-m-cresol | Butyl 4-hydroxybenzoate and salts thereof |
| 5-pyrimidinamine, 1,3-bis(2- | Propyl 4-hydroxybenzoate and salts thereof |
| ethylhexyl)hexahydro-5-methyl | |
| 5-bromo-5-nitro-1,3-dioxane | 3,3′-Dibromo-4,4′-hexamethylene |
| | dioxydibenzamidine and salts thereof |
| Bronopol | Thiomersal |
| 2,4-dichlorobenzyl alcohol | Phenylmercuric salts |
| 1-(4-chlorophenyl)-3-(3,4- | Undec-10-enoic acid and salts thereof |
| dichlorophenyl)urea | |
| Chlorocresol | N,N-dimethyl-N-[2-[2-[4-(1,1,3,3- |
| | tetramethylbutyl)phenoxy]ethoxy]ethyl]chloride |
| 5-chloro-2-(2,4-dichlorophenoxy)phenol | Ethyl Lauroyl Arginate HCl |
| Chloroxylenol | 1,2,3-propanetricarboxylic acid, 2-hydroxy, |
| | monohydrate |
| N,N″-methylenebis[N′-{3-(hydroxymethyl)- | 1,2,3-propantricarboxylic acid, 2-hydroxy- |
| 2,5-dioxoimidazolidin-4-yl}urea] | silver(1+) salt, monohydrate |
| Poly(hexamethylenebiguanide) | Silver citrate |
| hydrochloride | |
| 2-Phenoxyethanol | Benzenemethanaminium |
| Methenamine | |

Preferably, the composition is in the form of a semi-solid. As used herein, the term “semi-solid” means that the composition is in the form of a gel or “jelly” that can substantially sustain its shape when unsupported by external means. In other words, the composition may be a viscoelastic composition that is able to sustain its physical shape when unsupported. However, whilst the composition may be unsupported by external packaging or the like, the composition is sufficiently gel-like (i.e. viscoelastic) or soft that the composition may be applied directly to the teeth or skin of a user, and/or a portion of the composition may be taken from the product as a whole with the user's fingers or a separate applicator, such as a toothbrush, without requiring a breaking or snapping of the product.

As used herein, any reference to the term “jelly” refers to the meaning ascribed to said word in the United Kingdom; i.e. to a dessert prepared using fruit and sugar. This is known as “Jello” in the United States of America. As used herein, the terms “jelly-like” or “Jello-like” describe a composition that has a similar texture and structure to jelly or Jello desserts.

As used herein, the term “by weight of the composition” means that the percentage is given by weight of the total composition.

It has been found by the present inventors that by providing a composition comprising the specific components of the present invention, namely sorbitol and/or xylitol, a gelling agent and water in an amount of from about 20% to about 60% by weight of the composition, it is possible to include relatively high amounts of water in the composition whilst still maintaining a resistance to microbial growth without the use of undesirable preservatives. Without wishing to be bound, the inclusion of a gelling agent may help to further absorb the free water such that a higher content of water and/or fruit/vegetable juice may be included in the composition without requiring the addition of preservatives.

Furthermore, it has been found by the present inventors that by including a gelling agent, it is also possible to include these relatively high amounts of water in the composition whilst providing a viscoelastic semi-solid composition that can substantially sustain its shape when unsupported by external means. It is also possible to provide a composition that is sufficiently solid to substantially sustain its shape when unsupported by external means, whilst also being sufficiently jelly-like or soft that the user can remove a portion of the product without breaking or snapping said product as a whole. For the reasons identified above, the provision of a semi-solid composition may be advantageous in that it may obviate the need for any external packaging which is damaging to the environment.

The present invention therefore relates to a composition (preferably a semi-solid composition) that is free from preservatives, and yet is still resistant to microbial growth. The composition provides the user with a desirable product that is capable of being stored at room temperature and that may have a shelf-life of from six weeks to twenty months. Due to its self-preserving nature, there may be no need to store the composition in a refrigerator in order to prolong shelf-life. Typically, the product will be packaged in a pot or bottle with a label noting when the product was made, and advising when it should be used by.

Preferably, the composition (such as the cosmetic composition) is for use as an oral care product, for example as a composition for cleaning teeth (i.e. a toothpaste replacement). Alternatively or in addition, the composition (such as the cosmetic composition) may be for use as a skin care agent, such as for use as a skin cleanser and/or moisturiser and/or exfoliator.

Preferably, the composition (such as the cosmetic composition) is a semi-solid composition for use as an oral care agent. Application of the product may be effected as for any conventional toothpaste. For example, once a suitable size portion of the product has been taken from the composition using a toothbrush, said portion is then applied to the teeth of the user using the toothbrush. Optionally, said portion may be mixed with a bit of water and then applied to the teeth of the user. The user may then brush their teeth using the toothbrush having the product thereon, and after a couple/several minutes, the product may then be rinsed off the teeth using water.

For ease of reference, these and further aspects of the present invention are now discussed under appropriate section headings. However, the teachings under each section are not necessarily limited to each particular section.

### BRIEF DESCRIPTION OF FIGURES

Embodiments of the present invention are described, by way of example only, with reference to the accompanying figures in which:

FIG. 1 shows a composition prepared in accordance with Example 1.

FIG. 2 shows a composition prepared in accordance with Example 2.

FIG. 3 shows a composition prepared in accordance with Comparative Example 1.

FIG. 4 shows a composition prepared in accordance with Comparative Example 2.

FIG. 5 shows a composition prepared in accordance with Comparative Example 3.

### DETAILED DESCRIPTION

### Composition

As discussed herein, in one aspect of the invention, there is provided a composition comprising

(i) a sugar alcohol selected from the group consisting of sorbitol, xylitol, and mixtures thereof;

(ii) water in an amount of from about 20% to about 60% by weight of the total composition; and

(iii) a gelling agent.

As described above, the composition is preferably in the form of a semi-solid. Therefore, in some embodiments, external packaging is not required to maintain the shape of the composition. However, external packaging such as a pot or container may still of course be utilised in order for convenience of storage or to further increase the shelf-life of the product and reduce risk of spoilage, or where it may be required for hygienic purposes.

### Sugar Alcohol

As discussed herein, the composition comprises a sugar alcohol selected from sorbitol, xylitol, and mixtures thereof.

In some embodiments, the sugar alcohol is or comprises sorbitol. In some embodiments, the sugar alcohol is sorbitol.

In some embodiments, the sugar alcohol is or comprises xylitol. In some embodiments, the sugar alcohol is xylitol.

In some embodiments, the sugar alcohol is a combination of sorbitol and xylitol.

In some embodiments, the total combined amount of sorbitol and xylitol (such as the total amount of sorbitol) is from about 5% to about 70% by weight of the composition. In some embodiments, the total combined amount of sorbitol and xylitol (such as the total amount of sorbitol) is from about 10% to about 60% by weight of the composition. In some embodiments, the total combined amount of sorbitol and xylitol (such as the total amount of sorbitol) is from about 15% to about 55% by weight of the composition. In some embodiments, the total combined amount of sorbitol and xylitol (such as the total amount of sorbitol) is from about 20% to about 50% by weight of the composition. In some embodiments, the total combined amount of sorbitol and xylitol (such as the total amount of sorbitol) is from about 25% to about 45% by weight of the composition. In some embodiments, the total combined amount of sorbitol and xylitol (such as the total amount of sorbitol) is from about 25% to about 40% by weight of the composition. In some embodiments, the total combined amount of sorbitol and xylitol (such as the total amount of sorbitol) is from about 25% to about 35% by weight of the composition.

In some preferred embodiments, the total combined amount of sorbitol and xylitol (such as the total amount of sorbitol) is from about 10% to about 50% by weight of the composition.

In some embodiments, the amount of sorbitol is from about 10% to about 50% by weight of the composition. In some embodiments, the amount of sorbitol is from about 20% to about 40% by weight of the composition. In some embodiments, the amount of sorbitol is from about 25% to about 30% by weight of the composition

In some embodiments, the total combined amount of sorbitol and xylitol (such as the total amount of a combination of sorbitol and xylitol) is from about 5% to about 70% by weight of the composition. In some embodiments, the total combined amount of sorbitol and xylitol (such as the total amount of a combination of sorbitol and xylitol) is from about 10% to about 60% by weight of the composition. In some embodiments, the total combined amount of sorbitol and xylitol (such as the total amount of a combination of sorbitol and xylitol) is from about 15% to about 55% by weight of the composition. In some embodiments, the total combined amount of sorbitol and xylitol (such as the total amount of a combination of sorbitol and xylitol) is from about 20% to about 50% by weight of the composition. In some embodiments, the total combined amount of sorbitol and xylitol (such as the total amount of a combination of sorbitol and xylitol) is from about 25% to about 45% by weight of the composition. In some embodiments, the total combined amount of sorbitol and xylitol (such as the total amount of a combination of sorbitol and xylitol) is from about 25% to about 40% by weight of the composition. In some embodiments, the total combined amount of sorbitol and xylitol (such as the total amount of a combination of sorbitol and xylitol) is from about 25% to about 35% by weight of the composition. In some embodiments, the total combined amount of sorbitol and xylitol (such as the total amount of a combination of sorbitol and xylitol) is from about 30% to about 35% by weight of the composition.

In some preferred embodiments, the total combined amount of sorbitol and xylitol (such as the total amount of a combination of sorbitol and xylitol) is from about 10% to about 50% by weight of the composition.

As used herein, the term “total combined amount of sorbitol and xylitol” refers to the amount of the sugar alcohol in the composition, wherein the sugar alcohol is selected from the group consisting of sorbitol, xylitol, and mixtures thereof. This term therefore encompasses embodiments in which the composition comprises only sorbitol or xylitol as the sugar alcohol with the amount of the other sugar alcohol being 0%.

In some embodiments, the sugar alcohol is a combination of sorbitol and xylitol. In such embodiments, the sorbitol and xylitol may be present in a weight ratio of from about 50:1 to about 1:20, such as from about 40:1 to about 1:10, such as from about 30:1 to about 1:5, such as from about 25:1 to about 1:1, such as from about 25:1 to about 5:1, such as from about 20:1 to about 10:1. In some embodiments, the sorbitol and xylitol may be present in a weight ratio of from about 50:1 to about 10:1. In some embodiments, the amount of sorbitol present in the composition may be greater than the amount of xylitol.

It was surprisingly found by the present inventors that the combination of at least one of these specific sugar alcohols with water and a gelling agent provided a composition that may be prepared in the form of a semi-solid as defined above. For example, the composition may be prepared in the form of a jelly-like composition (“Jello-like” in the USA). The provision of such a semi-solid composition may be advantageous as it may enable the composition to be stored without requiring the use of packaging. Indeed, when the product is an oral care agent, the provision of the composition in such a semi-solid form may obviate the need for a conventional toothpaste tube, which are typically made of plastic and/or metal and thus are not environmentally friendly.

It was found by the present inventors that the inclusion of at least one of these two sugar alcohols was required in order to enable the preparation of such a semi-solid composition that could sustain its physical shape when unsupported by external packaging, but which was sufficiently soft that the user could shape the composition and also could remove some of the composition by dipping their toothbrush into it. As detailed in the experimental section, erythritol, for example, was found to provide a low-viscosity gel, which could not be regarded as semi-solid as it would not sustain its shape when unsupported by external means.

In contrast to sugar, the sugar alcohol of the present invention does not have a detrimental effect on oral health, but conversely can have positive antimicrobial effect. Furthermore, the literature has shown that xylitol can have remineralising effect in combination with other active oral care materials.

### Water

As discussed herein, the composition comprises water in an amount of from about 20% to about 60% by weight of the composition. The water may be provided by any suitable liquid or aqueous source. The water may be provided by distilled water, tap water, rain water, sea water, plant infusions, decoctions (such as tea and/or coffee), alcohol solutions (such as beer, wine and/or spirits), vinegar, fruit juice, vegetable juice or mixtures thereof. Preferably, the water is provided by distilled water, tap water, rain water, sea water, plant infusions, or mixtures thereof. Preferably, the water is provided by distilled water, tap water, rain water, plant infusions, or mixtures thereof. Preferably, the water is provided by distilled water, tap water, or mixtures thereof. The water may also be provided as a dilution agent in any commercially available surfactant and/or humectant products that may be utilised in the composition.

For example, in embodiments in which the composition is an oral care agent, the water may be provided by distilled water, tap water, rain water, or mixtures thereof.

In some embodiments, the water (such as distilled water, tap water, rain water, or mixtures thereof) is present in an amount of from about 25% to about 60% by weight of the composition. In some embodiments, the water (such as distilled water, tap water, rain water, or mixtures thereof) is present in an amount of from about 20% to about 55% by weight of the composition. In some embodiments, the water (such as distilled water, tap water, rain water, or mixtures thereof) is present in an amount of from about 20% to about 50% by weight of the composition. In some embodiments, the water (such as distilled water, tap water, rain water, or mixtures thereof) is present in an amount of from about 20% to about 45% by weight of the composition. In some embodiments, the water (such as distilled water, tap water, rain water, or mixtures thereof) is present in an amount of from about 25% to about 45% by weight of the composition. In some embodiments, the water (such as distilled water, tap water, rain water, or mixtures thereof) is present in an amount of from about 25% to about 40% by weight of the composition. In some embodiments, the water (such as distilled water, tap water, rain water, or mixtures thereof) is present in an amount of from about 25% to about 35% by weight of the composition. The total amount of water in the composition includes the amount of water included as a dilution agent in any of the ingredients utilised in the compositions, such as for example any commercially available surfactant and/or humectant products that may be utilised in the composition.

It was found by the present inventors that it was possible to include water in such relatively high amounts without also requiring the use of preservatives in order to maintain a shelf-life of several weeks when the water was combined with the gelling agent and sugar alcohol as described herein. Furthermore, it was found by the present inventors that the inclusion of water in an amount of from 20% to 60% by weight of the composition enables a composition to be prepared that is semi-solid as defined above. As such, a composition may be prepared that is sufficiently solid that it is able to sustain its physical shape when unsupported by external means, but which is sufficiently soft that the user may apply the composition to an applicator (e.g. a finger, toothbrush or other cosmetic applicator) simply by dipping the applicator into the composition and scooping a portion of the composition onto the applicator. In some embodiments, the inclusion of water in the amounts defined herein enables the preparation of a composition in the form of a jelly-like substance.

### Gelling Agent

As discussed herein, the composition comprises a gelling agent. The gelling agent may be selected from any suitable gelling agent. In particular, the gelling agent may be selected from any gelling agent that allows for the product to set in the form of a semi-solid when the product is prepared via a cold process and/or a hot process (i.e. with or without applying heat during preparation of the product). Preferably, the gelling agent may be selected from any gelling agent that allows for the product to set in the form of a semi-solid when the product is prepared via a hot process (i.e. by applying heat to the mixture during preparation of the product).

In some embodiments, the gelling agent is a hydrocolloid gelling agent. As the skilled person will appreciate, hydrocolloids are a heterogeneous group of long chain polymers characterised by their property of forming viscous dispersions and/or gels when dispersed in water. Some hydrocolloids have the ability to modify the rheology of compositions comprising water. However, there are two distinct types of hydrocolloid: gelling agents (i.e. those that modify the texture and/or solidity of the composition) and thickening agents (i.e. those that modify the viscosity of the composition). The water-thickening property is common to all hydrocolloids, whilst only a few hydrocolloids have the ability to form gels (i.e. to act as a gelling agent). Gel formation is typically regarded as the phenomenon involving the association or cross-linking of the polymer chains to form a three-dimensional network that traps or immobilises the water within it to form a rigid structure that is resistant to flow. In other words, the composition becomes viscoelastic exhibiting both the characteristics of a liquid and a solid. In contrast, thickening of a composition merely involves the non-specific entanglement of conformationally disordered polymer chains, and is essentially a polymer-solvent interaction (see, for example, D. Saha and S. Bhattacharya, J. Food Sci. Technol., 2010, 47 (6): 597-597).

Gels may be defined as a form of matter intermediate between solid and liquid, and which show mechanical rigidity. They comprise polymer molecules cross-linked to form a tangled and interconnected molecular network immersed in a liquid medium. The word “gel” typically refers to high moisture compositions that substantially sustain their physical shape when released from their container. More specifically, gels are viscoelastic systems with a storage modulus (G′) larger than the loss modulus (G″). Though all hydrocolloids may thicken aqueous dispersions, only a comparatively few gums are understood to form gels. Hydrocolloids that may act as gelling agents include modified starch, agar, carrageenan, pectin, gellan gum, alginate (when combined with calcium ions), methyl cellulose, and hydroxylpropyl methylcellulose. Hydrocolloids such as xanthan gum, guar gum, konjac gum, gum tragacanth and gum Arabic are known thickening agents, but are not generally regarded as being gelling agents.

In some embodiments, the gelling agent may be selected from the group consisting of carrageenan, agar, pectin, a combination of a salt of alginic acid and calcium ions, calcium alginate, gellan gum, methyl cellulose, hydroxypropyl methylcellulose, modified starch, and mixtures thereof. In some embodiments, the gelling agent may be selected from the group consisting of carrageenan, agar, pectin, a combination of a salt of alginic acid and calcium ions, calcium alginate, gellan gum, and mixtures thereof. The salt of alginic acid may be selected from sodium alginate, potassium alginate, ammonium alginate, magnesium alginate, and mixtures thereof. In some embodiments, the salt of alginic acid is sodium alginate, which is combined with a source of calcium ions in order to form the gel. As used herein, the term “agar” can be used interchangeably with “agar agar”.

As the skilled person will appreciate, carrageenan is traditionally split into three basic forms: lambda-carrageenan, kappa-carrageenan and iota-carrageenan. The structures of lambda-, kappa-, and iota-carrageenan are shown below:

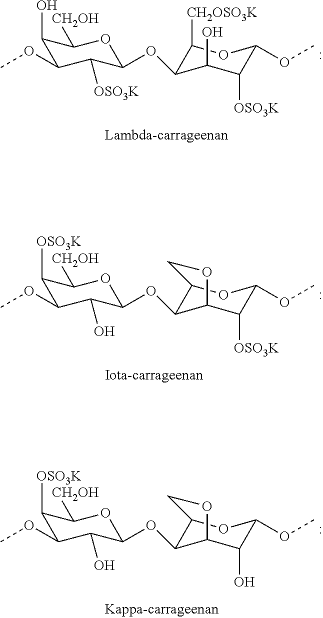

It has been found by the present inventors that kappa-carrageenan and/or iota-carrageenan may provide for an improved gelling effect as compared with that of lambda-carrageenan. Therefore, in some embodiments, the carrageenan is kappa-carrageenan or iota-carrageenan. Therefore, in some embodiments, the gelling agent is selected from the group consisting of kappa-carrageenan, iota-carrageenan, agar, pectin, a combination of a salt of alginic acid and calcium ions, calcium alginate, gellan gum, and mixtures thereof.

In some embodiments, the gelling agent is selected from the group consisting of carrageenan, agar, pectin, gellan gum, and mixtures thereof. In some embodiments, the gelling agent is selected from the group consisting of iota-carrageenan, kappa-carrageenan, agar, pectin, gellan gum, and mixtures thereof.

In some embodiments, the gelling agent is or comprises iota-carrageenan. In some embodiments, the gelling agent is or comprises kappa-carrageenan. In some embodiments, the gelling agent is or comprises agar.

In some embodiments, the gelling agent is or comprises gellan gum. The gellan gum may be high acyl gellan gum and/or low acyl gellan gum. As the skilled person will appreciate, gellan gum is a straight chain polymer that comprises repeating units of glucose, rhamnose and glucuronic acids. “High acyl” gellan gum has two acyl substituents, acetate and glycerate, that are located on the glucose residues. On average, there is one glycerate per repeat unit and one acetate every two repeat units when both are located on the same glucose residue.

Deacylated (or “low acyl”) gellan gum has these acyl groups removed during processing. In some embodiments, the gelling agent is or comprises high acyl gellan gum. In some embodiments, the gelling agent is or comprises low acyl gellan gum.

As described hereinabove, the term “gelling agent” as used herein does not encompass hydrocolloids that act as thickening agents, but which do not enable the formation of viscoelastic gels. Therefore, as used herein, the term “gelling agent” does not encompass hydrocolloids such as xanthan gum, guar gum, konjac gum, gum tragacanth and gum Arabic. For example, xanthan gum is understood to be a thickening agent that thickens compositions when added during cold processing (i.e. when heat is not applied). However, xanthan gum is understood to not provide any additional gelling effect when a composition is produced by hot and/or cold processes.

Without being bound by theory, it has been found that kappa-carrageenan may form more rigid and brittle gels which have a tendency to spontaneously release fluids (syneresis) under certain conditions. On the other hand, iota-carrageenan has been found to form a soft and elastic gel which does not present such syneresis. It is thus particularly preferred if the gelling agent comprises or consists of iota-carrageenan. In some embodiments, the gelling agent consists of iota-carrageenan; i.e. iota-carrageenan is the only gelling agent used in the composition.

In some embodiments, the gelling agent is selected from the group consisting of iota-carrageenan, kappa-carrageenan, agar, gellan gum, and mixtures thereof. In some embodiments, the gelling agent is selected from the group consisting of iota-carrageenan, kappa-carrageenan, agar, and mixtures thereof.

In some embodiments, the gelling agent consists of only one gelling agent, such as for example one of iota-carrageenan, kappa-carrageenan, agar, pectin, a combination of a salt of alginic acid and calcium ions, calcium alginate or gellan gum. In some embodiments, the gelling agent comprises a combination of two or more gelling agents, such as two or more gelling agents selected from the group consisting of iota-carrageenan, kappa-carrageenan agar, pectin, a combination of a salt of alginic acid and calcium ions, calcium alginate and gellan gum. In some embodiments, the gelling agent is a combination of iota- and kappa-carrageenan. In some embodiments, the gelling agent is a combination of carrageenan and gellan gum, such as a combination of iota-carrageenan and gellan gum. In some embodiments, the gelling agent is a combination of carrageenan and agar, such as a combination of iota-carrageenan and agar. In some embodiments, the gelling agent is a combination of carrageenan and pectin, such as a combination of iota-carrageenan and pectin. In some embodiments, the gelling agent is a combination of agar and gellan gum.

In some embodiments, the gelling agent is a combination of carrageenan and agar. In some embodiments, the gelling agent is a combination of iota-carrageenan and agar. In some embodiments, the gelling agent is a combination of kappa-carrageenan and agar.

In some embodiments, the gelling agent (such as iota-carrageenan, kappa-carrageenan and/or agar) is present in an amount of no greater than about 5% by weight of the composition, such as in an amount of no greater than about 4% by weight of the composition, such as in an amount of no greater than about 3% by weight of the composition, such as in an amount of no greater than 2% by weight of the total composition, such as in an amount of no greater than about 1.5% by weight of the composition.

In some preferred embodiments, the gelling agent (such as iota-carrageenan, kappa-carrageenan and/or agar) is present in an amount of from about 0.01% to about 5% by weight of the composition. In some preferred embodiments, the gelling agent (such as iota-carrageenan, kappa-carrageenan and/or agar) is present in an amount of from about 0.1% to about 4% by weight of the composition. In some preferred embodiments, the gelling agent (such as iota-carrageenan, kappa-carrageenan and/or agar) is present in an amount of from about 0.5% to about 3% by weight of the composition. In some preferred embodiments, the gelling agent (such as iota-carrageenan, kappa-carrageenan and/or agar) is present in an amount of from about 0.5% to about 2% by weight of the composition. In some preferred embodiments, the gelling agent (such as iota-carrageenan, kappa-carrageenan and/or agar) is present in an amount of from about 1% to about 2% by weight of the composition.

In some embodiments, the gelling agent is a combination of carrageenan (e.g. kappa- and/or iota-carrageenan) and agar. The carrageenan and agar may be present in a weight ratio of from about 10:1 to about 1:10, such as from about 5:1 to about 1:5, such as from about 4:1 to about 1:4, such as from about 3:1 to about 1:3, such as from about 2:1 to about 1:2, such as about 1:1. The carrageenan and agar may be present in a weight ratio of from about 10:1 to about 1:2, such as from about 5:1 to about 1:1, such as from about 4:1 to about 1:1, such as from about 3:1 to about 1:1, such as from about 2:1 to about 1:1.

### Humectant

In some embodiments, the composition further comprises a humectant. The humectant is preferably provided in liquid form. The humectant may be selected from suitable humectants. As used herein, the term “humectant” does not encompass sugar alcohols selected from the group consisting of sorbitol, xylitol, and mixtures thereof. Therefore, any reference to “humectant” as used herein shall be taken to mean a humectant in addition to the sugar alcohol that is included as an essential feature of the present invention.

In some embodiments, the humectant is selected from the group consisting of honey, glycerine, monopropylene glycol, dipropylene glycol, 1,3-propanediol, butanediol, agave nectar, fruit syrups, herbal syrups, golden syrup, sugar solutions, and mixtures thereof. In some embodiments, the humectant is selected from honey, glycerine, monopropylene glycol, dipropylene glycol, 1,3-propanediol, butanediol, and mixtures thereof. Preferably, the humectant is selected from glycerine, monopropylene glycol, and mixtures thereof.

In some embodiments, the composition comprises humectant (such as glycerine, monopropylene glycol, and mixtures thereof) in an amount of at least about 1% by weight of the composition. In some embodiments, the composition comprises humectant (such as glycerine, monopropylene glycol, and mixtures thereof) in an amount of at least about 5% by weight of the composition. In some embodiments, the composition comprises humectant (such as glycerine, monopropylene glycol, and mixtures thereof) in an amount of at least about 10% by weight of the composition. In some embodiments, the composition comprises humectant (such as glycerine, monopropylene glycol, and mixtures thereof) in an amount of at least about 15% by weight of the total composition.

In some embodiments, the composition comprises humectant (such as glycerine, monopropylene glycol, and mixtures thereof) in an amount of from about 1% to about 50% by weight of the composition. In some embodiments, the composition comprises humectant (such as glycerine, monopropylene glycol, and mixtures thereof) in an amount of from about 5% to about 40% by weight of the composition. In some embodiments, the composition comprises humectant (such as glycerine, monopropylene glycol, and mixtures thereof) in an amount of from about 10% to about 30% by weight of the composition. In some embodiments, the composition comprises humectant (such as glycerine, monopropylene glycol, and mixtures thereof) in an amount of from about 15% to about 25% by weight of the composition. In some embodiments, the composition comprises humectant (such as glycerine, monopropylene glycol, and mixtures thereof) in an amount of from about 15% to about 20% by weight of the composition.

### Inorganic Material

In some embodiments, the composition further comprises an inorganic material. The inorganic material may be any inorganic material suitable for use in a composition, such as a cosmetic composition.

In some embodiments, the inorganic material is an abrasive material. In some embodiments, the inorganic material is an abrasive material selected from the group consisting of clays, micas, zeolites, silica, carbonates, bicarbonates, phosphates, metal oxides, salts, and mixtures thereof. In some embodiments, the inorganic material is an abrasive material selected from the group consisting of clays, micas, zeolites, silica, bicarbonates, phosphates, metal oxides, salts, and mixtures thereof. In some embodiments, the inorganic material is an abrasive material selected from the group consisting of clays, micas, zeolites, silica, carbonates, bicarbonates, metal oxides, salts, and mixtures thereof. In some embodiments, the inorganic material is an abrasive material selected from the group consisting of silica, bentonite, calcium carbonate, sodium bicarbonate, hydrated aluminium oxide, diatomaceous earth, dicalcium phosphate, hydroxyapatite, and mixtures thereof. In some embodiments, the inorganic material is an abrasive material selected from the group consisting of silica, bentonite, sodium bicarbonate, hydrated aluminium oxide, diatomaceous earth, hydroxyapatite, and mixtures thereof. In some embodiments, the inorganic material is an abrasive material selected from the group consisting of silica, bentonite, calcium carbonate, sodium bicarbonate, hydrated aluminium oxide, diatomaceous earth, and mixtures thereof. In some embodiments, the inorganic material is an abrasive material selected from the group consisting of silica, bentonite, hydrated aluminium oxide, and mixtures thereof. In some embodiments, the inorganic material is an abrasive material selected from the group consisting of silica, bentonite, and mixtures thereof.

In some embodiments, the inorganic material is or comprises silica. In some embodiments, the inorganic material is silica. The silica may preferably be hydrated silica (i.e. SiO₂.nH₂O, where n is an integer from 1 to 10).

In some embodiments, the inorganic material is or comprises dicalcium phosphate and/or hydroxyapatite. In some embodiments, the inorganic material is or comprises dicalcium phosphate. In some embodiments, the inorganic material is or comprises a combination of silica and dicalcium phosphate.

In some embodiments, the inorganic material is not or does not comprise calcium carbonate. In some embodiments, the inorganic material is not or does not comprise sodium bicarbonate. In some embodiments, the inorganic material is not or does not comprise dicalcium phosphate.

In some embodiments, the inorganic material is or comprises a clay. In some embodiments, the inorganic material is a clay. In such embodiments wherein the inorganic material is or comprises clay, the clay may be selected from suitable cosmetically acceptable clays. In some embodiments, the one or more clays is selected from the group consisting of bentone (hectorite), bentonite (such as sodium bentonite, potassium bentonite, calcium bentonite and/or aluminium bentonite), kaolin, talc, illite, fullers earth, Rhassoul/Ghassoul clay (Moroccan Lava Clay), smectite, montmorillonite, and mixtures thereof.

As the skilled person will appreciate, bentonite is an aluminium phyllosilicate clay comprising montmorillonite. In some embodiments, the inorganic material is or comprises a clay selected from the list consisting of sodium bentonite, potassium bentonite, calcium bentonite, aluminium bentonite, and mixtures thereof. In some embodiments, the inorganic material is or comprises sodium bentonite.

Kaolin refers to a clay mineral rich in the mineral kaolinite, a hydrated aluminium silicate with the chemical composition Al₂Si₂O₅(OH)₄. Kaolin clay may be known as White Clay, Rose Clay, China Clay, Light Kaolin, Sea Clay, Dead Sea Clay or mixtures thereof. For the purposes of this invention, the clay may be selected from any kaolin or kaolinite type clay.

Illite refers to a secondary mineral precipitate phyllosilicate or layered alumino-silicate. Illite has the general formula (K,H₃O)(Al,Mg,Fe)₂(Si,Al)₄O₁₀[(OH)₂,(H₂O)], but there may be considerable ion substitution. The illite clay may be selected from French red illite, French pink illite (a blend of red illite and White (kaolin) Clay), French green illite, French yellow illite and mixtures thereof.

As the skilled person will appreciate, talc refers to a clay mineral composed of hydrated magnesium silicate with the chemical formula H₂Mg₃(SiO₃)₄ or Mg₃Si₄O₁₀(OH)₂.

The skilled person understands that the term mica refers to silicate minerals, which are a type of phyllosilicate exhibiting a two-dimensional sheet or layer structure. The general formula for minerals of the mica group is XY₂₋₃Z₄O₁₀(OH, F)₂, where X is K, Na, Ba, Ca, Cs, Rb, H₃O, or NH₄; Y is Al, Mg, Fe²⁺, Li, Cr, Mn, V, or Zn; and Z is Si, Al, Fe³⁺, Be, or Ti. In some embodiments, the mica is a synthetic mica. In some embodiments, the one or more micas is a type of synthetic fluorphlogopite. Synthetic fluorphlogopite is known to be a synthetic mimic of a natural mineral that functions in cosmetics as a bulking agent and a viscosity increasing agent in aqueous solutions. Synthetic fluorphlogopite is partially composed of magnesium aluminium silicate sheets weakly bound together with potassium. The chemical formula of synthetic fluorphlogopite is Mg₃KAlF₂O(SiO₃)₃.

Zeolites are microporous aluminosilicate minerals. In some embodiments, the one or more zeolites may be selected from clinoptilolite (green zeolite clay), analcime, chabazite, heulandite, natrolite, phillipsite, stilbite and mixtures thereof.

As the skilled person appreciates, calamine is a combination of zinc oxide and iron oxide. Preferably, the calamine is provided in the form of a calamine powder.

Where the inorganic material is an abrasive material, it has been surprisingly found that it is possible to tailor the amount of abrasive material without negatively affecting the function of the composition. Indeed, the present inventors surprisingly found that any suitable amount of abrasive material can be included in the composition in order to provide the desired level of abrasivity without affecting the physical properties and/or function of the composition. Furthermore, it was surprisingly found that, even when the compositions comprise relatively high amounts of abrasive material (e.g. up to about 15% by weight), the total abrasivity of the composition was relatively low (with an RDA value of between 0 and 70) as compared with other known oral care compositions. This is desirable as it reduces the damage done to the user's teeth and gums when using the oral care agent, whilst still providing an oral care agent that cleans the user's teeth.

In some embodiments, the inorganic material may be a material that is suitable as an oral care agent. Examples of oral care agents may include abrasive materials as described hereinabove, such as silica, calcium carbonate, hydrated aluminium oxide, dicalcium phosphate, hydroxyapatite, and mixtures thereof. Alternatively or in addition, the inorganic material may comprise fluoride as an inorganic material. The fluoride may be provided by any suitable compound, such as for example sodium fluoride, stannous fluoride, olaflur, sodium monofluorophosphate, or mixtures thereof. In some embodiments, the inorganic material is or comprises a fluoride. In some embodiments, the inorganic material is or comprises sodium fluoride.

In some embodiments, the inorganic material is or comprises a combination of two or more oral care agents. In some embodiments, the inorganic material is or comprises a combination of an abrasive material in combination with a fluoride. In some embodiments, the inorganic material is or comprises a combination of component (a) and component (b), where component (a) is selected from the group consisting of silica, calcium carbonate, hydrated aluminium oxide, dicalcium phosphate, hydroxyapatite, and mixtures thereof, and component (b) is selected from the group consisting of sodium fluoride, stannous fluoride, olaflur, sodium monofluorophosphate, and mixtures thereof. In some embodiments, the inorganic material is or comprises a combination of component (a) and component (b), where component (a) is selected from the group consisting of silica, hydrated aluminium oxide, dicalcium phosphate, hydroxyapatite, and mixtures thereof, and component (b) is selected from the group consisting of sodium fluoride, stannous fluoride, olaflur, sodium monofluorophosphate, and mixtures thereof. In some embodiments, the inorganic material is or comprises a combination of silica and sodium fluoride. In some embodiments, the inorganic material is a combination of silica and sodium fluoride. In some embodiments, the inorganic material is or comprises a combination of dicalcium phosphate and sodium fluoride. In some embodiments, the inorganic material is a combination of dicalcium phosphate and sodium fluoride.

In some embodiments, the composition further comprises an inorganic material (such as an abrasive material, such as silica) in an amount of from about 1% to about 50% by weight of the total composition. In some embodiments, the composition further comprises an inorganic material (such as an abrasive material, such as silica) in an amount of from about 5% to about 40% by weight of the total composition. In some embodiments, the composition further comprises an inorganic material (such as an abrasive material, such as silica) in an amount of from about 5% to about 30% by weight of the total composition. In some embodiments, the composition further comprises an inorganic material (such as an abrasive material, such as silica) in an amount of from about 10% to about 20% by weight of the total composition. In some embodiments, the composition further comprises an inorganic material (such as an abrasive material, such as silica) in an amount of from about 10% to about 15% by weight of the total composition.

In some embodiments, the composition further comprises an inorganic material (such as an abrasive material, such as silica) in an amount of from about 1% to about 20% by weight of the total composition. In some embodiments, the composition further comprises an inorganic material (such as an abrasive material, such as silica) in an amount of from about 5% to about 15% by weight of the total composition.

In some embodiments, the composition comprises silica in an amount of from about 1% to about 20% by weight of the total composition, such as from about 5% to about 15% by weight of the total composition, such as from about 10% to about 15% by weight of the total composition.

The inorganic material (such as an abrasive material, such as silica) may be provided in powder form. Alternatively, the inorganic material may be provided as a colloidal dispersion.

It has been found by the present inventors that the inclusion of an inorganic material (such as an abrasive material, such as silica) may further reduce the inhibition of the growth of microorganisms, whilst also providing the composition with an active ingredient that may make said composition suitable for a particular cosmetic purpose, such as for use as an oral care agent. Without wishing to be bound, it is considered that the presence of the inorganic material (such as an abrasive material, such as silica) results in the dispersion of water within a colloidal matrix. This dispersion of the water across the matrix further inhibits the growth of microorganisms.

### Total Composition & Further Components

In some embodiments, the water and sugar alcohol (such as sorbitol) are present in a weight ratio of from about 10:1 to about 1:10, such as in a weight ratio of from about 5:1 to about 1:5, such as in a weight ratio of from about 2:1 to about 1:2, such as in a weight ratio of approximately 1:1. In some embodiments, the water and sugar alcohol are present in a weight ratio of from about 5:1 to about 1:1, such as in a weight ratio of from about 3:1 to about 1:1, such as in a weight ratio of from about 2:1 to about 1:1.

In some embodiments, the water and gelling agent (such as iota-carrageenan, kappa-carrageenan, agar, and mixtures thereof) are present in a weight ratio of from about 100:1 to about 10:1, such as in a weight ratio of from about 90:1 to about 15:1, such as in a weight ratio of from about 80:1 to about 20:1, such as in a weight ratio of from about 70:1 to about 20:1, such as in a weight ratio of from about 60:1 to about 20:1, such as in a weight ratio of from about 50:1 to about 20:1, such as in a weight ratio of from about 40:1 to about 20:1, such as in a weight ratio of from about 30:1 to about 25:1. In some embodiments, the water and gelling agent (such as iota-carrageenan, kappa-carrageenan, agar, and mixtures thereof) are present in a weight ratio of from about 100:1 to about 20:1, such as in a weight ratio of from about 50:1 to about 20:1, such as in a weight ratio of from about 30:1 to about 25:1.

In some embodiments, the water and gelling agent (such as a combination of iota-carrageenan and agar) are present in a weight ratio of from about 100:1 to about 10:1, such as in a weight ratio of from about 90:1 to about 15:1, such as in a weight ratio of from about 80:1 to about 20:1, such as in a weight ratio of from about 70:1 to about 20:1, such as in a weight ratio of from about 60:1 to about 20:1, such as in a weight ratio of from about 50:1 to about 20:1, such as in a weight ratio of from about 40:1 to about 20:1, such as in a weight ratio of from about 30:1 to about 25:1. In some embodiments, the water and gelling agent (such as a combination of iota-carrageenan and agar) are present in a weight ratio of from about 100:1 to about 20:1, such as in a weight ratio of from about 50:1 to about 20:1, such as in a weight ratio of from about 30:1 to about 25:1.

In some embodiments, the sugar alcohol (such as sorbitol or a combination of sorbitol and xylitol) and gelling agent (such as iota-carrageenan, kappa-carrageenan, agar, and mixtures thereof) are present in a weight ratio of from about 100:1 to about 20:1, such as in a weight ratio of from about 50:1 to about 20:1, such as in a weight ratio of from about 30:1 to about 25:1.

In some embodiments, the composition comprises:

(i) a sugar alcohol selected from the group consisting of sorbitol, xylitol, and mixtures thereof;

(ii) water in an amount of from about 20% to about 60% by weight of the composition; and

(iii) a gelling agent selected from the group consisting of carrageenan, agar, pectin, gellan gum, and mixtures thereof.

In some embodiments, the composition comprises:

(i) a sugar alcohol selected from the group consisting of sorbitol, xylitol, and mixtures thereof, wherein the total combined amount of sorbitol and xylitol is from about 20% to about 50% by weight of the composition;

(ii) water in an amount of from about 20% to about 60% by weight of the composition; and

(iii) a gelling agent in an amount of from about 0.01% to about 5% by weight of the composition.

In some embodiments, the composition comprises:

(i) a sugar alcohol selected from the group consisting of sorbitol, xylitol, and mixtures thereof, wherein the total combined amount of sorbitol and xylitol is from about 20% to about 50% by weight of the composition;

(ii) water in an amount of from about 20% to about 60% by weight of the composition; and

(iii) a gelling agent selected from the group consisting of carrageenan, agar, pectin, gellan gum, and mixtures thereof, wherein the gelling agent is present in an amount of from about 0.01% to about 5% by weight of the composition.

In some embodiments, the composition comprises:

(i) a sugar alcohol selected from the group consisting of sorbitol, xylitol, and mixtures thereof, wherein the total combined amount of sorbitol and xylitol is from about 20% to about 50% by weight of the composition;

(ii) water in an amount of from about 20% to about 60% by weight of the composition;

(iii) a gelling agent selected from the group consisting of—carrageenan, agar, pectin, gellan gum, and mixtures thereof, wherein the gelling agent is present in an amount of from about 0.01% to about 5% by weight of the composition; and

(iv) a humectant selected from the group consisting of glycerine, monopropylene glycol, and mixtures thereof, wherein the humectant is present in an amount of from about 5% to about 30% by weight of the composition.

In some embodiments, the composition comprises:

(i) a sugar alcohol selected from the group consisting of sorbitol, xylitol, and mixtures thereof;

(ii) water in an amount of from about 20% to about 60% by weight of the composition;

(iii) a gelling agent; and

(iv) silica.

In some embodiments, the composition comprises:

(i) a sugar alcohol selected from the group consisting of sorbitol, xylitol, and mixtures thereof, wherein the total combined amount of sorbitol and xylitol is from about 20% to about 50% by weight of the composition;

(ii) water in an amount of from about 20% to about 60% by weight of the composition;

(iii) a gelling agent selected from the group consisting of—carrageenan, agar, pectin, gellan gum and mixtures thereof, wherein the gelling agent is present in an amount of from about 0.01% to about 5% by weight of the composition; and

(iv) silica in an amount of from about 5% to about 15% by weight of the composition.

In addition to the above, the composition may further comprise one or more cosmetically acceptable additives. The person skilled in the art is aware of a range of cosmetically acceptable additives which are suitable for incorporation into such compositions. In some embodiments, the one or more cosmetically acceptable additives are selected from a surfactant, starch, binder, filler, opacifier, UV absorbing material, exfoliating material, essential oil, vitamin, perfume, fragrance, flavouring agent, colouring, vegetable butter, vegetable oil, cocoa powder, arrowroot powder, fruit and/or herb extract, decorative item, sweetener, and mixtures thereof.

Fragrances or flavouring agents may be added to the product to make the experience of using the present composition more pleasant. Combining essential oils such as lavender, chamomile, menthol or rose absolute into fragrances or flavouring agents for the invention ensures the user has a pleasant experience.

In some embodiments, the composition thus further comprises a fragrance or flavouring agent. The amount of fragrance or flavouring agent is preferably from about 0.01% to about 10% by weight of the total composition, such as from about 0.1% to about 5% by weight of the total composition, such as from about 0.1% to about 4% by weight of the total composition, such as from about 0.5% to about 5% by weight of the total composition, such as from about 1% to about 5% by weight of the total composition, such as from about 0.5% to about 4% by weight of the total composition, such as from about 0.5% to about 3% by weight of the total composition, such as from about 0.5% to about 2% by weight of the total composition, such as from about 0.5% to about 1.5% by weight of the total composition.

The essential oils may be selected based on the fragrance or flavour desired, skin type to be treated and other effects desired based on the well-known properties of essential oils. The addition of essential oils, when taken in to the nose, are known to alter mood. For example, essential oils are known to create effects of drowsiness or stimulating the senses. Many well documented effects can be achieved by the use of essential oils. Likewise, if the composition is an oral care agent, the addition of essential oils can impart a pleasant sensation in the mouth.

In one embodiment, the one or more essential oils present in the product are selected from tea tree, tarragon, lemon myrtle, jasmine, ylang ylang, labdanum, lemongrass, rose otto, grapefruit, patchouli, rosemary, armoise, lemon, neroli, sweet violet, lavender, orange, vanilla, corn mint, peppermint, benzoin, hydrangea, litsea cubeba, cardamom, clove, wintergreen, myrrh, eucalyptus and chamomile blue. In one embodiment, the one or more essential oils present in the product are selected from tea tree, corn mint, peppermint, tarragon, lemon myrtle, labdanum, and lemon. In one embodiment, the one or more essential oils present in the composition comprises menthol. In one embodiment, the essential oil is tea tree oil.

Vitamins, particularly B, C and E are very beneficial for the skin. Vitamin rich ingredients such as Wheatgerm oil can also be used to deliver vitamins on to the skin. In one embodiment, the vitamins are selected from vitamin B, vitamin C, vitamin E and mixtures thereof. It will be appreciated by one skilled in the art that the vitamin may be provided from any suitable source. For example the vitamin(s) may be provided from a synthetic source or from incorporation into the product of a material, such as a natural material, that has a high vitamin content.

In some embodiments, the composition further comprises a surfactant. As used herein, the surfactant may be in addition to any “inorganic material” present; i.e. the terms “surfactant” and “inorganic material” are intended to be used to define separate and distinct components. Preferably, the surfactant is selected from the group consisting of acyl sarcosines, acyl sarcosinates, sodium lauryl sulfate, sodium laureth sulfate, sodium methyl cocoyl taurate, sodium dodecyl sulfate, sodium cocosulfate, ammonium lauryl sulfate, ammonium laureth sulfate, sodium myreth sulfate, disodium laureth sulfosuccinate, sodium lauryl sulfoacetate, lauryl betaine, cocamidopropyl betaine, trimethyl glycine betaine, sodium cocoamphoacetate, disodium cocoamphodiacetate, sodium lauroamphoacetate, disodium lauroamphodiacetate, cocoamidopropyl hydroxysultaine, cetrimonium chloride, behentrimonium chloride, and mixtures thereof. Preferably, the surfactant is selected from the group consisting of sodium lauroyl sarcosinate, N-lauroyl sarcosine, sodium lauryl sulfate, sodium laureth sulfate, sodium methyl cocoyl taurate, sodium dodecyl sulfate, sodium cocosulfate, ammonium lauryl sulfate, ammonium laureth sulfate, sodium myreth sulfate, disodium laureth sulfosuccinate, sodium lauryl sulfoacetate, lauryl betaine, cocamidopropyl betaine, trimethyl glycine betaine, sodium cocoamphoacetate, disodium cocoamphodiacetate, sodium lauroamphoacetate, disodium lauroamphodiacetate, cocoamidopropyl hydroxysultaine, cetrimonium chloride, behentrimonium chloride, and mixtures thereof. In some embodiments, the composition further comprises a surfactant selected from the group consisting of sodium lauroyl sarcosinate, N-lauroyl sarcosine, sodium lauryl sulfate, sodium laureth sulfate, sodium methyl cocoyl taurate, sodium cocosulfate, lauryl betaine, cocamidopropyl betaine, sodium cocoamphoacetate, and mixtures thereof. In some embodiments, the composition further comprises a surfactant selected from the group consisting of sodium lauroyl sarcosinate, sodium methyl cocoyl taurate, sodium cocosulfate, lauryl betaine, cocamidopropyl betaine, and mixtures thereof. In some embodiments, the composition further comprises a surfactant selected from the group consisting of sodium methyl cocoyl taurate, sodium lauroyl sarcosinate, cocamidopropyl betaine, and mixtures thereof. In some embodiments, the composition further comprises sodium lauroyl sarcosinate, cocamidopropyl betaine, and mixtures thereof. In some embodiments, the composition further comprises sodium lauroyl sarcosinate. In some embodiments, the composition further comprises cocamidopropyl betaine. In some embodiments, the composition further comprises sodium methyl cocoyl taurate.

In some embodiments, the composition further comprises a surfactant (such as sodium lauroyl sarcosinate, N-lauroyl sarcosine, sodium lauryl sulfate, sodium laureth sulfate, sodium methyl cocoyl taurate, sodium cocosulfate, lauryl betaine, cocamidopropyl betaine, sodium cocoamphoacetate, or mixtures thereof) in an amount of from about 0.1% to about 40% by weight, such as from about 0.5% to about 30% by weight of the composition, such as from about 1% to about 20% by weight of the composition, such as from about 1.5% to about 15% by weight of the composition, such as from about 2% to about 10% by weight of the composition, such as from about 2.5% to about 5% by weight of the composition. As used herein, the amount of surfactant relates to the total amount of active surfactant in the composition (i.e. not including the amount of dilution agent in any surfactant products utilised in the composition).

The advantages of the present invention discussed herein may be applied to any product, for example an oral care product. Other categories that would be applicable under the present invention include:
shaving preparations, shower gels & shower jellies, moisturisers, skincare & body lotions, face and/or hair masks, sunscreen products, shampoos, conditioners & hair dressings and lip balms.

All are categories of product, which conventionally require packaging, are stored at room temperature and have a long shelf-life.

Materials which may be included in the present composition include but are not limited to: Oil, Butter & Waxes—such as almond oil, sesame oil, evening primrose oil, jojoba oil, cocoa butter, shea butter, mango butter, cupuacu butter, lanolin, beeswax, rose wax, orange peel wax—if included, typically used in a range of from about 5% to about 70% by weight of the composition.

Capsules—such as a combination of a salt of alginic acid (such as sodium alginate) and a calcium salt (such as calcium lactate) to prepare a capsule dispersed in the composition—if included, typically used in an amount of from about 0.1% to about 10% by weight of the composition.

Whole Fruits & Vegetables—such as avocado, banana, strawberries, blueberries—if included, typically used in a range of from about 0.5% to about 25% by weight of the composition.

Fragrance materials—if included, typically used in a range of from about 0.1% to about 5% by weight of the composition.

Flavouring agent—if included, typically used in a range of from about 0.1% to about 10% by weight of the composition.

Colorant materials—if included, typically used in a range of from about 0.001% to about 2% by weight of the composition.

Sunscreens (UV absorbing and/or reflecting materials)—such as octocrylene, titanium dioxide, ethylhexyl methoxycinnamate, sulisobenzone and salts thereof (such as benzophenone-4 and/or benzophenone-5), octyl methoxycinnamate, butylmethoxydibenzoylmethane, homosalate, ecamsule, and mixtures thereof—if included, typically used in a range of from about 1% to about 25% by weight of the composition.

Sea salt—if included, typically used in a range of from about 0.1% to about 20% by weight of the composition.

Herbs, Cereals, Plant Materials and Beans—such as oats, rice, cinnamon, vanilla, adzuki beans, coffee beans, seaweeds, ground bamboo, cocoa powder, arrowroot powder—if included, typically used in a range of from about 0.01% to about 15% by weight of the composition.

Starches—such as corn starch, potato starch, tapioca starch, rice starch, and mixtures thereof—if included, typically used in a range of from about 0.1 to about 15% by weight of the composition

Protein Sources—such as tofu, banana, soya, soya lecithin, eggs—if included, typically used in a range of from about 1% to about 30% by weight of the composition.

Decorative Items—glitter, paper such as rice paper, sequins, popping candy, dried or fresh flowers, dried or fresh fruit, herbs, vegetables, parts thereof or mixtures thereof—if included, typically used in a range of from about 0.1% to about 15% by weight of the composition.

Sweeteners—such as acesulfame K, aspartame, saccharin, sucralose, stevia, or mixtures thereof—if included, typically used in a range of from about 0.01% to about 5% by weight of the composition.

Charcoal—if included, typically in a range of from about 0.1% to about 10% by weight of the composition.

The above ranges provide preferred amounts of each of the components. Each of these ranges may be taken alone or combined with one or more other component ranges to provide a preferred aspect of the invention.

### Process

As discussed herein, according to a second aspect of the invention, there is provided a process for the production of a composition comprising (i) a sugar alcohol selected from the group consisting of sorbitol, xylitol, and mixtures thereof; (ii) water in an amount of from about 20% to about 60% by weight of the composition; and (iii) a gelling agent; the process comprising the following steps:
(a) preparing a preparatory composition comprising water and a sugar alcohol selected from the group consisting of sorbitol, xylitol, and mixtures thereof;
(b) contacting the preparatory composition with a gelling agent; and
(c) introducing the resultant composition to a mould.

In some embodiments, the composition produced by this process is a composition as defined hereinabove.

In some embodiments, the process comprises a step (a-i) heating the preparatory composition to a temperature of at least about 60° C. before and/or during and/or after step (b) of contacting the preparatory composition with a gelling agent. In some embodiments, the process comprises a step (a-i) heating the preparatory composition to a temperature of at least about 60° C. before step (b) of contacting the preparatory composition with a gelling agent. In some embodiments, the preparatory composition is heated in step (a-i) to a temperature of from about 65° C. to about 95° C., such as from about 70° C. to about 90° C., such as from about 75° C. to about 85° C. before and/or during and/or after step (b) of contacting the preparatory composition with a gelling agent.

In some embodiments, step (b) thus comprises contacting a gelling agent with the heated preparatory composition, wherein the preparatory composition has been heated to a temperature of at least about 60° C. Such process is a “hot process” in which the gelling agents interacts with a heated preparatory composition to provide the gelling effect. In some embodiments, step (b) comprises contacting a gelling agent with the heated preparatory composition, wherein the preparatory composition has been heated to a temperature of from about 60° C. to about 100° C., such as from about 65° C. to about 95° C., such as from about 70° C. to about 90° C., such as from about 75° C. to about 85° C.

In some embodiments, the preparatory composition is heated in step (a-i) to a temperature of no greater than about 100° C., such as no greater than about 95° C., such as no greater than about 90° C., such as no greater than about 85° C., such as no greater than about 80° C. In some embodiments, the preparatory composition is heated in step (a-i) to a temperature of from about 80° C. to about 90° C.

Therefore, in some embodiments, the process comprises the following steps:
(a) preparing a preparatory composition comprising water and a sugar alcohol selected from the group consisting of sorbitol, xylitol, and mixtures thereof;
(a-i) heating the preparatory composition to a temperature of at least about 60° C.;
(b) contacting the heated preparatory composition with a gelling agent; and
(c) introducing the resultant composition to a mould.

In some embodiments, the resultant composition (i.e. the preparatory composition that has been contacted with the gelling agent) is heated to or (when the process comprises step (a-i) of heating the preparatory composition) is maintained at a temperature of at least about 60° C. for at least about 1 minute prior to and/or after introduction of the resultant composition to a mould. In some embodiments, the resultant composition is heated to or is maintained at a temperature of at least about 60° C. for at least about 5 minutes, such as at least about 10 minutes, such as from about 10 minutes to about 30 minutes, such as from about 10 minutes to about 15 minutes. In some embodiments, the resultant composition is stirred during this period of heating. In some embodiments, the resultant composition is heated to or is maintained at a temperature of from about 60° C. to about 100° C., such as from about 65° C. to about 95° C., such as from about 70° C. to about 90° C., such as from about 75° C. to about 85° C. for a period of at least about 1 minute.

In some embodiments, the process further comprises step (d) of allowing the resultant composition to cool. In some embodiments, the process further comprises step (d) of cooling the resultant composition. Preferably, if the process involves the step (a-i) of heating the preparatory composition, then the process further comprises step (d) of allowing the resultant composition to cool. Preferably, if the process involves the step (a-i) of heating the preparatory composition, then the process further comprises step (d) of cooling the resultant composition.

Therefore, in some embodiments, the process comprises the following steps:
(a) preparing a preparatory composition comprising water and a sugar alcohol selected from the group consisting of sorbitol, xylitol, and mixtures thereof;
(a-i) heating the preparatory composition to a temperature of at least about 60° C.;
(b) contacting the heated preparatory composition with a gelling agent;
(c) introducing the resultant composition to a mould; and
(d) allowing the resultant composition to cool.

The water may first be heated prior to the addition of water to the preparatory composition. Therefore, in some embodiments, the process further comprises a step of heating water, optionally together with one or more cosmetically acceptable additives, prior to the addition of water to the preparatory composition in step (a). In some embodiments, the water is heated prior to the addition of water to the preparatory composition in step (a) to a temperature of at least about 80° C., such as at least about 90° C., such as at least about 95° C., such as at least about 100° C. In some embodiments, the water is heated prior to the addition of water to the preparatory composition in step (a) to a temperature of from about 70 to about 120° C., such as from about 80° C. to about 110° C., such as from about 90 to about 105° C., such as from about 90 to about 100° C., such as approximately 100° C. For example, the water may be heated to a temperature of from about 90 to about 110° C.

The water may be heated prior to the addition of water to the preparatory composition in step (a) together with one or more cosmetically acceptable additives. For example, the water may be heated together with a plant material (such as the petals, stem or leaves of a flower, coffee beans or powder, tea leaves, tea powder, or tea bags, herbs) to prepare a plant infusion or decoction. In some embodiments, the water is heated together with a plant material selected from rose petal, camomile (optionally dried), marigold (optionally dried), herbs, tea (such as green tea), coffee, and mixtures thereof.

Therefore, in some embodiments, the process comprises the following steps:

(0) heating water, optionally together with one or more cosmetically acceptable additives;

(a) preparing a heated preparatory composition comprising the heated water and a sugar alcohol selected from the group consisting of sorbitol, xylitol, and mixtures thereof;

(b) contacting the heated preparatory composition with a gelling agent; and

(c) introducing the resultant composition to a mould.

As discussed herein, the composition is preferably in the form of a semi-solid. Therefore, in some embodiments, the resultant composition is left to set (e.g. by cooling) in step (c) and/or (d) such that it sets to a semi-solid composition. This may be achieved by, for example, leaving the composition in the mould at room temperature until the composition is semi-solid, chilling the composition in the mould in a refrigerator until the composition is semi-solid, or any other method of cooling the composition.

In some embodiments, the process further comprises step (d) of allowing the resultant composition to cool such that it sets in the form of a semi-solid composition. In some embodiments, the process further comprises step (d) of cooling the resultant composition such that it sets in the form of a semi-solid composition.

As discussed herein, according to a third aspect of the invention, there is provided a product obtained or obtainable by a process for the production of a composition comprising (i) a sugar alcohol selected from the group consisting of sorbitol, xylitol, and mixtures thereof; (ii) water in an amount of from about 20% to about 60% by weight of the composition; and (iii) a gelling agent; the process comprising the following steps:
(a) preparing a preparatory composition comprising water and a sugar alcohol selected from the group consisting of sorbitol, xylitol, and mixtures thereof;
(b) contacting the preparatory composition with a gelling agent; and
(c) introducing the resultant composition to a mould.

The composition obtained or obtainable by the process as described herein is preferably a composition as defined hereinabove.

### Method

In one aspect of the invention, there is provided a method comprising contacting the teeth, hair and/or skin of a user with the composition as defined herein. Preferably, the method comprises contacting the teeth of a user with the composition. Preferably, the method is a cosmetic method comprising contacting the teeth, hair and/or skin of a user with the cosmetic composition as defined herein. Preferably, the method comprises contacting the teeth of a user with the cosmetic composition.

In some embodiments, the composition (such as cosmetic composition) is an oral care agent. Preferably, the composition is a composition for cleaning (e.g. brushing) the teeth of a user. The composition may be applied to the teeth of the user either directly, or after the user has taken a suitable sized, smaller portion of the composition from the total composition using their fingers or a separate applicator. For example, when the composition is a composition for cleaning the teeth of a user, the user may dip a toothbrush into the composition and scoop a portion of the composition onto the toothbrush for subsequent application to the teeth.

The composition may be applied to the teeth of the user as with a conventional toothpaste. In other words, the user may apply the composition to the teeth, brush their teeth so as to rub the composition onto and over the enamel, and then subsequently rinsing their mouth with water to remove the composition from the teeth. It has been surprisingly found by the present inventors that the composition as defined herein has a relatively low abrasivity (with an RDA value of between 0 and 70) even when including abrasive materials in an amount of up to about 15% by weight.

It has been surprisingly found by the present inventors that the composition described herein may be provided in a semi-solid form that is sufficiently solid to substantially sustain its physical shape without external packaging, but which is sufficiently soft or jelly-like that a portion of the product can be removed from the whole composition without breaking or snapping the composition (e.g. by scooping a portion of the product from the whole composition by the use of the user's fingers and/or a separate applicator).

### EXAMPLES

### Example 1

A product having the following composition was prepared:

| Raw Material Type | wt. % |
|---|---|
| Water | 27.00 |
| Sorbitol | 35.50 |
| Hydrated silica | 12.00 |
| Sodium lauroyl sarcosinate | 4.80 |
| Iota-carrageenan | 0.80 |
| Monopropylene glycol | 19.00 |
| Flavour | 0.90 |
| TOTAL | 100.00 |

The product was prepared using the following method:
1. The water was heated to about 100° C.;
2. The heated water was mixed together with the sorbitol, surfactant and silica to prepare a preparatory composition;
3. The gelling agent was mixed together with the monopropylene glycol;
4. The preparatory composition was then heated to a temperature of about 80° C., and the gelling agent/monopropylene glycol mixture slowly added;
5. The resultant composition was stirred, and the temperature raised to around 80° C. for about 10 to 15 minutes;
6. The mixture was then removed from the heat, poured into a mould and allowed to cool until it set into semi-solid form.

The product was found to have a semi-solid consistency such that it can be turned out of the mould easily, and can sustain its physical shape when unsupported by the mould or any external packaging. The user was able to readily apply some of the composition to a toothbrush by dipping the toothbrush into the composition and scooping a portion of the semi-solid gel onto the brush.

The product was found to be microbiologically stable during storage.

### Example 2

A product having the following composition was prepared:

| Raw Material Type | wt. % |
|---|---|
| Water | 27.00 |
| Xylitol | 35.50 |
| Hydrated silica | 12.00 |
| Sodium lauroyl sarcosinate | 4.80 |
| Iota-carrageenan | 0.80 |
| Glycerine | 19.00 |
| Flavour | 0.90 |
| TOTAL | 100.00 |

The method was the same as described in Example 1.

The product was found to have a semi-solid consistency such that it can be turned out of the mould easily, and can sustain its physical shape when unsupported by the mould or any external packaging. The user was able to readily apply some of the composition to a toothbrush by dipping the toothbrush into the composition and scooping a portion of the semi-solid gel onto the brush.

The product was found to be microbiologically stable during storage.

### Comparative Examples 1 to 3

The following compositions were prepared using the method described above in respect of Example 1. For the avoidance of doubt, it is noted that Comparative Examples 1 to 3 do not fall within the scope of the present invention.

TABLE 1
Comparative Examples 1 to 3

| | Formula (% by weight) | | |
|---|---|---|---|
| | Comp. | Comp. | Comp. |
| Raw Material Type | Example 1 | Example 2 | Example 3 |
| Water | 27.00 | 27.00 | 27.00 |
| Sorbitol | — | — | 35.50 |
| Erythritol | 35.50 | — | — |
| Isomalt | — | 35.50 | — |
| Sodium lauroyl sarcosinate | 4.80 | 4.80 | 4.80 |
| Hydrated Silica | 12.00 | 12.00 | 12.00 |
| Monoproylene glycol | 19.00 | 19.00 | 19.00 |
| Iota-carrageenan | 0.80 | 0.80 | — |
| Xanthan gum | — | — | 0.80 |
| Fragrance | 0.90 | 0.90 | 0.90 |
| TOTAL | 100.00 | 100.00 | 100.00 |

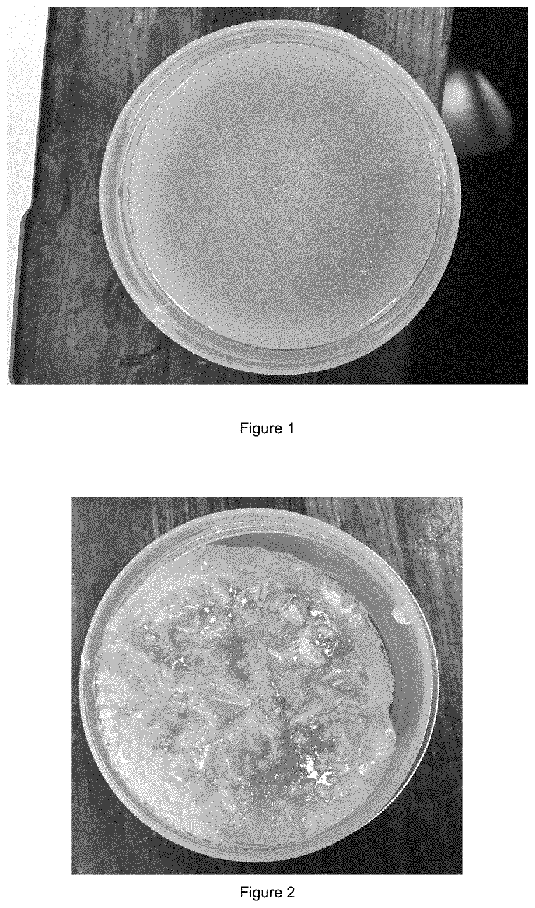

The properties of Examples 1 to 2 and Comparative Examples 1 to 3 were observed, and are recorded in the following Tables and shown in FIGS. 1 to 5:

TABLE 2
Observations on the properties of Examples 1 to 2

| Example | Observations |
|---|---|
| 1 | The product had the form of a jelly, which could be turned out from |
| | the mould and retained its shape when unsupported by any |
| | packaging. No crystals were formed in the composition. The |
| | product was sufficiently soft and jelly-like that the user was able to |
| | dip their toothbrush into the product and scoop a portion of the |
| | product onto the toothbrush for subsequent application to the teeth - see FIG. 1. |
| 2 | Same observations as for Example 1, noting that the composition |
| | was slightly stickier and tasted sweeter than Example 1. Slight |
| | crystallisation had occurred on the surface of the product; however |
| | the user was still able to scoop a portion onto a toothbrush with |
| | sufficient pay off due to the jelly-like structure of the composition - see FIG. 2. |

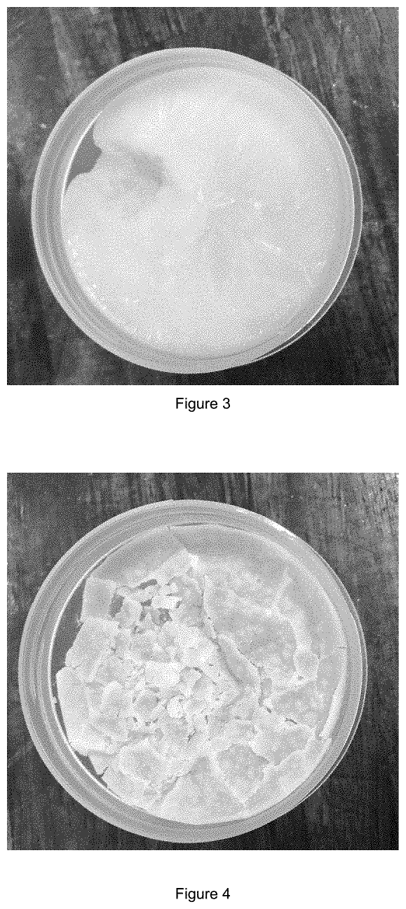

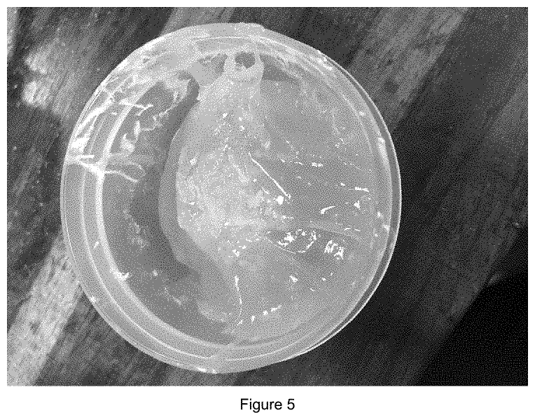

TABLE 3
Observations on the properties of Comparative Examples 1 to 3

| Comparative Example | Observations |
|---|---|
| 1 | The product did not form a jelly, but rather turned into a solid |
| | block of sugar alcohol crystals. The product could not be turned |
| | out from the mould whilst retaining the shape of the mould. |
| | There was also very little pay off when the user tried to scoop |
| | some of the product onto a toothbrush - see FIG. 3. |
| 2 | The product had the form a jelly and could be shaped into the |
| | form of the mould, and also be turned out from the mould whilst |
| | retaining the shape of the mould. However, the product had |
| | crystallised throughout and large crystals of sugar alcohol had |
| | formed across the surface of the jelly, which render the product |
| | unsuitable for use as a toothpaste - see FIG. 4. |
| 3 | The product did not form a jelly, but rather formed a sticky paste |
| | having a viscosity higher than a conventional liquid, but which |
| | was not a viscoelastic gel. It was not possible to shape the |
| | product into the form of the mould, and nor could the product be |
| | turned out whilst retaining the shape of the mould. Whilst the |
| | user was able to scoop some product onto a toothbrush by |
| | dipping the brush into the product, due to the high stickiness |
| | of the paste, the amount of product delivered to the toothbrush |
| | was much less than with the jelly-like product of Example 1, |
| | for example - see FIG. 5. |

### Example 3

A product having the following composition was prepared:

| Raw Material Type | wt. % |
|---|---|
| Water | 27.00 |
| Sorbitol | 35.50 |
| Hydrated silica | 12.00 |
| Lauroyl sarcosine | 4.80 |
| Agar agar | 0.80 |
| Monopropylene glycol | 19.00 |
| Flavour | 0.90 |
| TOTAL | 100.00 |

The method was the same as described in Example 1.

The product was found to have a semi-solid consistency, although the jelly was found to be slightly more brittle than the jelly texture of Examples 1 and 2. A thin layer of liquid was formed on the surface of the jelly. The user was able to apply a small amount of the composition to a toothbrush by dipping the toothbrush into the composition and scooping a portion of the semi-solid gel onto the brush.

### Example 4

A product having the following composition was prepared:

| Raw Material Type | wt. % |
|---|---|
| Water | 27.00 |
| Sorbitol | 35.50 |
| Hydrated silica | 12.00 |
| Lauroyl sarcosine | 4.80 |
| Iota-carrageenan | 0.40 |
| Agar agar | 0.40 |
| Monopropylene glycol | 19.00 |
| Flavour | 0.90 |
| TOTAL | 100.00 |

The method was the same as described in Example 1.

The product was found to have a semi-solid consistency (in the form of a jelly) such that it can be turned out of the mould easily, and can sustain its physical shape when unsupported by the mould or any external packaging. The user was able to readily apply some of the composition to a toothbrush by dipping the toothbrush into the composition and scooping a portion of the semi-solid gel onto the brush.

### Example 5

A product having the following composition was prepared:

| Raw Material Type | wt. % |
|---|---|
| Water | 27.00 |
| Sorbitol | 35.50 |
| Hydrated silica | 12.00 |
| Lauroyl sarcosine | 4.80 |
| Iota-carrageenan | 0.64 |
| Agar agar | 0.16 |
| Monopropylene glycol | 19.00 |
| Flavour | 0.90 |
| TOTAL | 100.00 |

The method was the same as described in Example 1.

The product was found to have a semi-solid consistency (in the form of a jelly) such that it can be turned out of the mould easily, and can sustain its physical shape when unsupported by the mould or any external packaging. The jelly was found to have a clear and translucent appearance. The user was able to readily apply some of the composition to a toothbrush by dipping the toothbrush into the composition and scooping a portion of the semi-solid gel onto the brush.

### Example 6

A product having the following composition was prepared:

| Raw Material Type | wt. % |
|---|---|
| Water | 27.00 |
| Sorbitol | 35.50 |
| Hydrated silica | 12.00 |
| Lauroyl sarcosine | 4.80 |
| Iota-carrageenan | 0.16 |
| Agar agar | 0.64 |
| Monopropylene glycol | 19.00 |
| Flavour | 0.90 |
| TOTAL | 100.00 |

The method was the same as described in Example 1.

The product was found to have a semi-solid consistency with a looser texture than that of Example 5. The user was able to readily apply some of the composition to a toothbrush by dipping the toothbrush into the composition and scooping a portion of the semi-solid gel onto the brush; the resulting composition on the brush of the toothbrush had a stickier texture than those of Examples 4 and 5.

### Example 7

A product having the following composition was prepared:

| Raw Material Type | wt. % |
|---|---|
| Water | 27.00 |
| Sorbitol | 35.50 |
| Hydrated silica | 12.00 |
| Lauroyl sarcosine | 4.80 |
| Xanthan Gum | 0.40 |
| Agar agar | 0.40 |
| Monopropylene glycol | 19.00 |
| Flavour | 0.90 |
| TOTAL | 100.00 |

The method was the same as described in Example 1.

The product was found to have a gel-like consistency. The gel-like composition was found to have a clear and translucent appearance. The user was able to apply some of the composition to a toothbrush by dipping the toothbrush into the composition and scooping a portion of the gel onto the brush.

### Example 8

A product having the following composition was prepared:

| Raw Material Type | wt. % |
|---|---|
| Water | 27.00 |
| Sorbitol | 35.50 |
| Hydrated silica | 12.00 |
| Lauroyl sarcosine | 4.80 |
| Xanthan Gum | 0.40 |
| Iota-carrageenan | 0.40 |
| Monopropylene glycol | 19.00 |
| Flavour | 0.90 |
| TOTAL | 100.00 |

The method was the same as described in Example 1.

The product was found to have a thick gel-like consistency and found to be fairly dense. The user was able to apply some of the composition to a toothbrush by dipping the toothbrush into the composition and scooping a portion of the gel onto the brush; the resulting composition on the brush of the toothbrush was found to have a stripy texture.

### Example 9

A product having the following composition was prepared:

| Raw Material Type | wt. % |
|---|---|
| Water | 27.00 |
| Sorbitol | 35.50 |
| Hydrated silica | 12.00 |
| Lauroyl sarcosine | 4.80 |
| Sodium alginate | 0.40 |
| Agar agar | 0.40 |
| Monopropylene glycol | 19.00 |
| Flavour | 0.90 |
| TOTAL | 100.00 |

The method was the same as described in Example 1.

The product was found to have a thick gel-like consistency with no liquid on the surface. The user was able to apply some of the composition to a toothbrush by dipping the toothbrush into the composition and scooping a portion of the gel onto the brush.

### Example 10

A product having the following composition was prepared:

| Raw Material Type | wt. % |
|---|---|
| Water | 27.00 |
| Sorbitol | 35.50 |
| Hydrated silica | 12.00 |
| Lauroyl sarcosine | 4.80 |
| Sodium alginate | 0.40 |
| Iota-carrageenan | 0.40 |
| Monopropylene glycol | 19.00 |
| Flavour | 0.90 |
| TOTAL | 100.00 |

The method was the same as described in Example 1.

The product was found to have a thick gel-like consistency. The user was able to apply some of the composition to a toothbrush by dipping the toothbrush into the composition and scooping a portion of the gel onto the brush.

### Example 11

A product having the following composition was prepared:

| Raw Material Type | wt. % |
|---|---|
| Water | 27.70 |
| Sorbitol | 35.50 |
| Hydrated silica | 12.00 |
| Lauroyl sarcosine | 4.80 |
| Iota-carrageenan | 0.05 |
| Agar agar | 0.05 |
| Monopropylene glycol | 19.00 |
| Flavour | 0.90 |
| TOTAL | 100.00 |

The method was the same as described in Example 1.

The product was found to have a smooth but more liquid texture than that of Example 4 to 6.

### Example 12

A product having the following composition was prepared:

| Raw Material Type | wt. % |
|---|---|
| Water | 23.80 |
| Sorbitol | 35.50 |
| Hydrated silica | 12.00 |
| Lauroyl sarcosine | 4.80 |
| Iota-carrageenan | 2.0 |
| Agar agar | 2.0 |
| Monopropylene glycol | 19.00 |
| Flavour | 0.90 |
| TOTAL | 100.00 |

The method was the same as described in Example 1.

The product was found to have a semi-solid consistency, which had a higher degree of solidity than the compositions of Examples 4 to 6.

Various modifications and variations of the present invention will be apparent to those skilled in the art without departing form the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

1. A composition comprising
(i) a sugar alcohol selected from the group consisting of sorbitol, xylitol, and mixtures thereof;
(ii) water in an amount of from about 20% to about 60% by weight of the composition; and
(iii) a gelling agent.

2. The composition according to claim 1, wherein the total combined amount of sorbitol and xylitol is from about 10% to about 50% by weight of the composition.

3. The composition according to claim 1, wherein the sugar alcohol is sorbitol.

4. The composition according to claim 1, wherein the sugar alcohol is a combination of sorbitol and xylitol present in a weight ratio of from about 50:1 to about 10:1.

5. The composition according to claim 1, wherein the water is present in an amount of from about 25% to about 40% by weight of the composition, wherein the water is provided by distilled water, tap water, rainwater, sea water, fruit juice, vegetable juice or mixtures thereof.

6. (canceled)

7. The composition according to claim 1, wherein the gelling agent is present in an amount of from about 0.01% to about 5% by weight of the composition.

8. (canceled)

9. The composition according to claim 1, wherein the gelling agent is selected from the group consisting of carrageenan, agar, pectin, gellan gum, methyl cellulose, hydroxypropyl methylcellulose, a combination of sodium alginate and calcium ions, and mixtures thereof.

10. The composition according to claim 1, wherein the gelling agent is a combination of carrageenan and agar, wherein the carrageenan and agar are present in a weight ratio of from about 10:1 to about 1:2.

11. (canceled)

12. The composition according to claim 1, wherein the water and gelling agent are present in a weight ratio of from about 100:1 to about 10:1.

13. The composition according to claim 1, further comprising a humectant, wherein the humectant is selected from the group consisting of glycerine, monopropylene glycol, dipropylene glycol, 1,3-propanediol, butanediol, and mixtures thereof.

14. The composition according to claim 13, wherein the humectant is present in an amount of from about 5% to about 30% by weight of the composition.

15. (canceled)

16. The composition according to claim 1, further comprising a surfactant, wherein the surfactant is selected from the group consisting of sodium lauroyl sarcosinate, N-lauroyl sarcosine, sodium lauryl sulfate, sodium laureth sulfate, sodium methyl cocoyl taurate, sodium dodecyl sulfate, sodium cocosulfate, ammonium lauryl sulfate, ammonium laureth sulfate, sodium myreth sulfate, disodium laureth sulfosuccinate, sodium lauryl sulfoacetate, lauryl betaine, cocamidopropyl betaine, trimethyl glycine betaine, sodium cocoamphoacetate, disodium cocoamphodiacetate, sodium lauroamphoacetate, disodium lauroamphodiacetate, cocoamidopropyl hydroxysultaine, cetrimonium chloride, behentrimonium chloride, and mixtures thereof.

17. The composition according to claim 16, wherein the surfactant is present in an amount of from about 0.1% to about 10% by weight of the composition.

18. (canceled)

19. The composition according to claim 1, further comprising an inorganic material, wherein the inorganic material is present in an amount of from about 1% to about 20% by weight of the composition.

20. (canceled)

21. The composition according to claim 19, wherein the inorganic material is silica.

22. The composition according to claim 1, further comprising one or more cosmetically acceptable additives selected from a binder, filler, opacifier, exfoliating material, vegetable butter, vegetable oil, essential oil, vitamin, perfume, fragrance, colouring, protein, decorative item, flavouring agent, acidity modifier, herb and/or herb extract, fruit and/or fruit extract, botanical extract, and mixtures thereof.

23. The composition according to claim 1, wherein the composition is a semi-solid composition.

24. The composition according to claim 1, wherein the composition is an oral care composition.

25. A process for production of a composition as defined in claim 1, the process comprising the steps of:
(a) preparing a preparatory composition comprising water and a sugar alcohol selected from the group consisting of sorbitol, xylitol, and mixtures thereof;
(b) contacting the preparatory composition with a gelling agent; and
(c) introducing the resultant composition to a mould.

26. The process according to claim 25, wherein the process comprises the steps of:
(a) preparing a preparatory composition comprising water and a sugar alcohol selected from the group consisting of sorbitol, xylitol, and mixtures thereof;
(a-i) heating the preparatory composition to a temperature of from about 60° C. to about 100° C.;
(b) contacting the heated preparatory composition with a gelling agent; and
(c) introducing the resultant composition to a mould;
(d) allowing the resultant composition to cool.

27-29. (canceled)
